(19) United States
(12) Patent Application Publication
(10) Pub. No.: US 2008/0228288 A1
(43) Pub. Date: Sep. 18, 2008

(54) Composite Prosthetic Foot

(76) Inventors: Ronald Harry Nelson, Salt Lake City, UT (US); Brian T. Coop, Arlington, VA (US); Gerald Stark, Chattanooga, TN (US); Daniel Buck, Salt Lake City, UT (US)

Correspondence Address: DOUGLAS T. JOHNSON;MILLER & MARTIN, 1000 VOLUNTEER BUILDING, 832 GEORGIA AVENUE, CHATTANOOGA, TN, 37402-2289, US

(21) Appl. No.: 12/046,253

(22) Filed: Mar. 11, 2008

Related U.S. Application Data
(60) Provisional application No. 60/906,687, filed on Mar. 13, 2007.

(51) Int. Cl. A61F 2/66

(52) U.S. Cl. 623/55; 623/53

(57) ABSTRACT

A prosthetic foot is provided with an ankle plate for attachment to a lower leg prosthesis and supporting a composite frame having a hollow composite biasing structure, preferably in the form of a generally helical spring curved about a vertical axis.

The present application claims priority to the Mar. 13, 2007 filing date of U.S. provisional patent application, Ser. No. 60/906,687.

### FIELD OF THE INVENTION

The present invention relates to a prosthetic foot and specifically to a prosthetic foot formed of composite tubing.

### BACKGROUND OF THE INVENTION

Prostheses for amputated feet have been used since the times of ancient civilization. However, over time there have been numerous refinements in medicine, surgery, technology and prosthetic science resulting in the development of many designs of prosthetic feet. These feet can generally be divided into five categories:
SACH foot, an acronym for Solid Ankle Cushion Heel, this is a simple design that is relatively inexpensive, durable, and can be made in lightweight configurations.
SAFE FOOT—Solid Ankle Flexible Endoskeleton, this foot is more flexible than the SACH foot by allowing for a smooth rollover while walking and is sometimes called a flexible keel foot.
Single Axis feet contain an ankle joint which allows the foot to flex or rotate up and down (plantar/dorsal flexion). This flexion allowed by the ankle joint increases knee stability during early stance phase in gait.
Multiple Axis feet not only contain an ankle joint, allowing the foot to flex up and down, but can also flex from side to side (inversion/eversion).
Energy Storing or Dynamic Response feet absorb energy within their structure when the foot contacts the ground and release that energy late in stance to provide forward propulsion.

The basic functions designed to be accomplished by a prosthetic foot are to provide a stable weight-bearing surface, to absorb shock, to replace lost motor function, to replicate the anatomic joint, and to restore cosmetic appearance. The SACH foot mimics ankle plantar flexion with a compliant heel pad which allows for a smooth gait. The single axis foot adds passive plantar flexion and dorsal flexion which increases stability during stance phase. Both the multi-axis foot and the dynamic response foot are energy storing designs. The multi-axis foot adds inversion/eversion to the plantar flexion and dorsal flexion provided by the single axis design with a mechanism that dissipates a substantial amount of the energy input during gait. The multi-axial foot also handles uneven terrain by allowing the foot to conform to the surface while continuing to provide a stable platform for weight bearing. Dynamic response feet utilize a basic metal, nylon or composite leaf spring to store and release energy during gait and are particularly useful for amputees with a very active lifestyle. As the amputee's cadence or activity level increases, more spring comes into play resulting in a greater push off. Some of the most widely recognized commercial embodiments of dynamic response feet include Flexfoot by Ossur, Springlite by Otto Bock, Seattle feet by Seattle Systems and Carbon Copy by Ohio Willow Wood.

The functions of the foot and ankle during gait are numerous and subtle, so that during the initial phase of heel strike the foot absorbs impact through controlled plantar flexion allowing the foot to be flat on the ground shortly after heel strike. Following this there is controlled dorsal flexion coupled with inversion and eversion to cope with irregular terrain. Then the gait proceeds to the rollover phase with the foot deforming during the single-limb stance, transitioning from a flexible shock absorber to a rigid platform for pushing. During late stance phase immediately preceding toe-off there is plantar flexion and power generation. Finally, during swing phase, dorsal flexors are active lifting the toes to prevent toe stubbing and possible stumbling.

Many dynamic response foot prostheses have been created and introduced for use by amputees. However, the performance characteristics desired by each amputee vary substantially and a tunable dynamic response prosthetic foot design providing high and low dynamic response capabilities is desired.

In particular, most dynamic response prosthetic feet, typified by those disclosed in the Van Phillips patents, utilize a J-shaped spring and rely upon the length of the spring element for energy storage. The result is that most dynamic response prostheses are of a high profile design and not really suitable for foot replacement at the ankle. Therefore, a need exists for a lightweight, low profile foot prosthesis providing energy storing dynamic response, plantar/dorsal flexion and inversion/eversion.

An autoclave manufacturing process is utilized on most current composite construction dynamic response prosthetic feet. This process uses a single sided tool to produce components which are generally planar in nature. The shapes are usually gently curved in only one primary direction. The autoclave process is expensive and slow and is unsuited for the manufacture of hollow shapes with a complex geometry. The material near the mid-plane of this planar structure are relatively inefficient, contributing weight but not capable of storing significant flexural energy. Most dynamic response prosthetic feet today are of relatively simple construction, being essentially planar in direction. Such feet are generally loaded almost exclusively in flexure. Delamination failures occasionally occur in current dynamic response prosthetic foot designs when the structure is loaded in a way to incur interlaminar tensile stresses or when interlaminar shear stresses exceed the strength of the relatively weak matrix material, usually epoxy resin, such as when a curved section in the foot is loaded so as to open or flatten or flatten the curve. This delamination occurs because there are no fibers oriented in the direction of the tensile or shear load. Current autoclave construction processes are not conducive to the construction of structures which can place fibers in the direction where these tensile or shear delamination type loads are transmitted.

It is therefore an object of the invention to produce an improved prosthetic foot of hollow composite tubing at reasonable cost, with high strength, great reliability, high level of compliance and terrain conformance.

It is an additional object of the invention to produce a prosthetic foot of hollow composite tubing in a fashion allowing a wide range of geometries to be utilized effectively in foot structure, while providing a relatively light-weight foot capable of supporting and storing high torsional and radial tensile loads with fibers oriented in a way to avoid large interlaminar tensile or shear stresses.

### SUMMARY OF THE INVENTION

In order to provide low profile dynamic response foot prostheses, the present invention comprises a mounting element such as an ankle plate adapted for attachment to a lower leg pylon and composite fiber tubing forming a rear spring element. Preferably the tubing may form a helical spring, and the foot may also have a forward flexible keel portion fabricated of composite tubing. The tubing might also form a forward frame or posterior heel structure, the heel typically is a reverse design from the forward frame.

### BRIEF DESCRIPTION OF THE DRAWINGS

A more complete appreciation of the invention and the advantages is obtained with reference to the following detailed description considered in connection with the accompanying drawings wherein:

FIG. 1 is a side plan view of a prosthetic foot made with composite tubular springs according to the invention;

FIG. 2 is a top plan view of the prosthetic foot of FIG. 1;

FIG. 3 is a front plan view of the prosthetic foot of FIG. 1;

FIG. 4 is a perspective view of an alternative prosthetic foot made with composite tubular spring elements having a generally rectangular cross section;

FIG. 5 is a perspective view of a prosthetic foot made with composite tubular helical springs having a generally rectangular cross section;

FIG. 6 is a side perspective view of a prosthetic foot made with composite helical tubular springs according to the present invention with a resilient foot plate;

FIG. 7 is a side perspective view of a prosthetic foot made with a telescoping helical composite tubular spring;

FIG. 8 is a side perspective view of a prosthetic foot made with composite tubular helical springs having a lateral axis rectangular cross section;

FIG. 9 is an alternative construction of a prosthetic foot made with composite tubular springs having a rectangular cross section and wire length substantially aligned in the direction of plantar, flexion and dorsal;

FIG. 10A is a side plan view of a prosthetic foot made with an S-shaped composite tubular spring having a generally rectangular cross section;

FIG. 10B is a top perspective view of the prosthetic foot of FIG. 10A;

FIG. 11A is a front perspective view of a prosthetic foot made with pairs of composite tubes extending to the front and rear; and

FIG. 11B is a rear perspective view of the prosthetic foot of FIG. 11A.

### DETAILED DESCRIPTION OF THE INVENTION

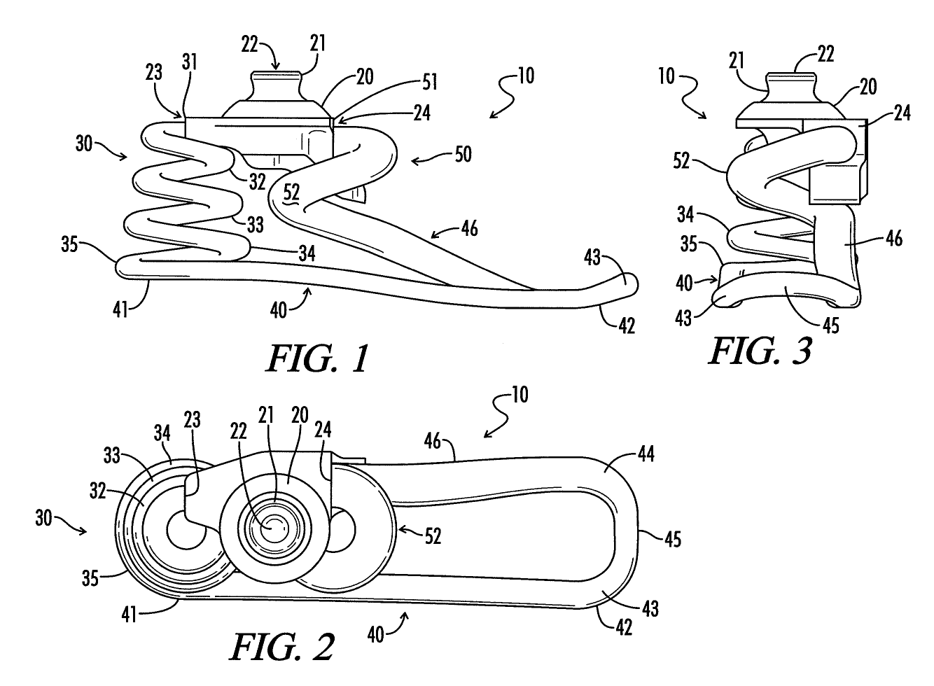

Referring now to the drawings wherein like numbered reference numerals designate corresponding parts throughout the several views, according to the embodiments of the invention illustrated in the non-limiting FIGS. 1 through 11. The principal elements of prosthetic foot 10 comprise a mounting element such as an ankle plate 20, a heel spring 30, a keel portion formed of a forespring 50 and an arch member 46, and a lateral longitudinal support member 40. The ankle plate 20 has an upward facing attachment point defined by a housing such as the convex hemispherical surface beneath an inverted pyramid 21 which is received by a mating component attached to the end of a pylon or other attachment extending downward from amputee's stump. These mating surfaces allow for static multiaxial alignment of the foot with the remainder of the prosthesis and the limb. The aperture 22 is solely provided for weight reduction. There is a rear attachment point 23 for connection with heel spring 30 and a forward attachment point 24 for attachment to the forespring 50. In the illustrated embodiment of ankle plate 20, the rear 23 and fore 24 attachment points are shown in a horizontal configuration to receive the proximate end 31 of heel spring 30 and distal end 51 of forespring 50. The attachment points 23,24 may also be oriented in a vertical direction so that the proximal 31 and distal 51 ends are upwardly facing. Vertical connections are less desirable due to additional manufacturing concerns required by the additional upward bend in the tubes, the creation of additional stress points at the bend, and the resulting higher profile of the prosthesis.

Turning then to the heel spring 30, this element extends from proximal end 31 through first spring loop 32, second spring loop 33 and third spring loop 34 to distal end 35. The term “wire” is commonly used in the spring industry to refer to solid or hollow finger-like members of a spring. All of the wire sections of heel spring 30 are preferably hollow. In some cases, the hollow area may be quite small on the order of only three-hundredths of an inch, and in other cases the hollow area may be relatively large having a diameter on the order of 0.5 inches, or equal to as much as 90 percent of the outer wire diameter. Similarly, wall thicknesses may be relatively thin on the order of three or four-hundredths of an inch, or much thicker and very nearly equal to the radius of the wire section. The heel spring 30 is preferably manufactured from long composite fibers such as carbon, Kevlar, or fiberglass preimpregnated with curable resin, which are wrapped around an inflatable bladder and placed within a mold. The bladder is then inflated and the mold is heated to a temperature sufficient to melt the resin and activate the curing process. This forms the composite fibers into a circle or other tubular shape and this configuration permits the composite material to handle sheer stresses very effectively. Indeed there is little or no sheer stress between layers of fiber in a hollow composite tube. The result is a stiff tubular frame that is extraordinarily light. The diameter of the composite tube, the cross-sectional shape of the tube, the thickness and number of layers of composite material utilized and the composition of the composite materials utilized may be altered to achieve optimum performance characteristics. The result is a heel spring adequate to support a 1200 pound load, as is representative of the load that may be placed on the foot by a 300 pound amputee. It will be understood that many variations in the fore spring sound rear spring 30 are possible, and tubular “u” shaped spring elements are also useful in their prosthetic foot designs.

The preferred manufacturing technology to create the shaped hollow composite tubes utilizes matched female molds with an internal cavity forming the outer shape of the product. Resin impregnated fiber material is either placed in the tubular cavity or wrapped about an internal pressure bladder which is placed into the cavity. Several examples of this manufacturing technology are disclosed as used in various industries in U.S. Pat. Nos. 5,624,519; 6,340,509; 6,270,104; 6,143,236; 6,361,840; 5,692,970; 5,985,197; 6,248,024; 5,505,492; 5,534,203; and 6,319,346.

Many variations are possible in the manufacturing process of hollow composite tubing. For example, disentegratable core material may be used inside the bladder to rigidize the bladder, making it easier to place fiber materials on the bladder. The entire assembly, consisting of fiber overwrapping the bladder with an internal core may then be placed inside the mold, the mold can then be closed and heated, and air or other gas is used to pressure the bladder internally, compacting and applying pressure to the fiber resin composite structure. In addition, fiber material may also be placed directly on the tool mold cavity surfaces. Some fiber material could be placed in the tool and some material placed on the bladder.

Pre-impregnated fiber material is generally used, which has uncured epoxy resin already impregnated into the fiber. Dry fiber can also be used, such as woven or braided material. If dry materials are used, liquid epoxy resin can be injected during cure using an external pump or a transfer device inside the tool which forces a volume of resin to be moved from a precharged reservoir in the tool into the part during cure. Inflation of the internal pressure bladder can be coordinated with the resin injection in this case.

A preferred construction of composite fiber tubing utilizes unidirectional fiber oriented along the wire sections consisting of roughly 25% to 75% of the total laminate thickness. Additional layers of fiber are oriented at ±45° and at 90° to the wire center line. The fibers may also be oriented at other angles corresponding to the principle directions of stress within the structure. The use of ±45° fiber in the hollow tubing wall allows the springs to efficiently store, release and carry torsional loads. Prior art dynamic response prosthetic feet produced in autoclaves lack this ability and their geometries are significantly restricted.

The use of ±45° and 90° fiber orientation in the composite fiber tubing walls sections also greatly strengthens the resistance to delamination type forces. In sum, the use of hollow composite tubular walled wire sections containing ±45° and 90° fiber in the cross section walls allows the spring to become a torsional spring in some or all areas rather than a pure flexural spring as in prior art dynamic response feet. The ability to carry torsional loads allows a more complex geometry, which in turn allows designs to be developed with longer wire lengths. This allows greater compliance in the foot while minimizing breakage and delamination problems. The use of hollow cross sections also removes inefficient material from the prosthetic foot, reducing the weight of the foot. If a wide flat cross section is desired, multiple hollow cavities extending the length of the section may be utilized in what is referred to as a multi-celled hollow structure.

Prosthetic feet according to this invention will usually be designed to utilize torsional loading to store at least half of the energy load dynamics retained by the biasing elements of the shoe. It will also be understood that the hollow tubing may be filled with non-structural material for damping or to adjust the weight or profile of the foot.

The lateral longitudinal support 40 of the prosthetic foot begins at proximal end 41 formed with or attached to distal end 35 of heel spring 30 and extends through central length to distal end 42 at about corner 43 of front toe 45. From the toe 45, the illustrated composite tube structure rounds corner 44 to arch section 46 which extends to a first loop 52 of forespring 50. The lateral longitudinal support 40 is flexible, that flexibility being variable according to the diameter of the composite tube over the length from proximal end 41 to distal end 42 as well as the thickness, layers and composition of the composite materials are chosen for use and manufacture of this section of the foot 10. A foot plate, not shown, may be added to the lateral longitudinal support 40, or indeed, support 40 may be omitted or replaced by such a foot plate joining the heel and fore sections of the foot. The toe section 45 extending from the outer corner 43 to inner corner 44 helps adapt the front portion of the foot 10 to uneven terrain just as the distal end 35 of heel spring 30 helps conform the rear portion of the prosthetic foot 10 to uneven terrain. The arch 46 and forespring 50 form the keel of the foot and provide energy storage functionality improved over that accomplished by J-shaped leaf springs of higher profile prior art dynamic response foot prostheses and the rear spring 30 and forespring 50 allow for inversion/eversion motion as well as plantar and dorsal flexion. In the illustrated embodiment, the axis of both the heel spring 30 and forespring 50 is substantially vertical, and this dynamically stores the vertical impact of the amputee's weight as a torsional load. If the anticipated impact is more forward, as in the case of the running amputee, the axis of the forespring 50 might be adjusted to descend forward from the ankle plate 20.

The composite frame including heel spring 30, support 40, toe 45, arch 46 and forespring 50 are preferably manufactured in several pieces which are then attached together by polyacrylate or other secure adhesive resin. A frame made from two or three separate pieces would be a typical construction. When made from separate pieces, the individual pieces may be mixed and matched to an individual amputee's weight and mobility. In addition, a foot shell may be added over the composite framework to provide desirable cosmetic appearance and provide additional support for the prosthetic foot 10. The toe 45 of the composite frame is preferably positioned at about the location of the ball of the anatomical foot and it is contemplated that an add on foot shell will have a flexible toe forward of the frame toe 45.

In use, heel spring 30 bottoms out on itself instead of failing, and for this reason, it is desirable that the helical coils or loops 32,33,34 of heel spring 30 be relatively closely spaced. It may be noted that the circumference of loops 32,33,34 increases or telescopes outward as the heel spring 30 proceeds downward from attachment point 23 on ankle plate 20 to the distal end 35 of the heel spring 30. This increasing circumference may also be combined with increasing tubing diameter and the layering, thickness and composition of composite materials so that the compression of the heel spring will not be linear but may instead provide increasing resistance to compression. The presently preferred heel design has a smaller pitch for the first loop 32 and third loop 34 and a medium pitch for the middle loop 33. Alternative pitch modifications are possible for particular performance and characteristics. Ideally, the coils are pitched to close on each other before failure.

The helical structure of the spring 30 allows the efficient storage of torsional loads over a relatively long wire length. The cross section of the wire in the loops of the heel spring 30 may also vary to alter the compression profile of the spring.

Apart from changing composition of composite materials utilized, such as utilizing fiberglass for lower modulus and higher flexibility in portions of the composite frame, the fiber orientation may also be changed to provide additional strength in certain directions. For instance, the fibers are preferably aligned at about a 45 degree angle to the axis of the tubing to manage the torsional load in the helical spring portions 30,50 of the frame. It will also be seen that the forward arch 46 design accommodates the inclusion of an arch in a cosmetic foot shell and within a shoe worn over the foot 10 better than typical prior art dynamic response prosthetic feet. It is to be appreciated that the length of the spring, or the springs' effective wire length, the distance between attachment point 31 and distal end 35 of heel spring 30 and between attachment point 51 and inner corner 44 on the forespring 50 and arch 46 comprising the keel, are proportional to the amount of energy that the spring may store. By utilizing helical spring elements 30,50, additional effective length is added to the springs while providing relatively lower profile for the dynamic responsiveness or energy sharing capacity of the foot.

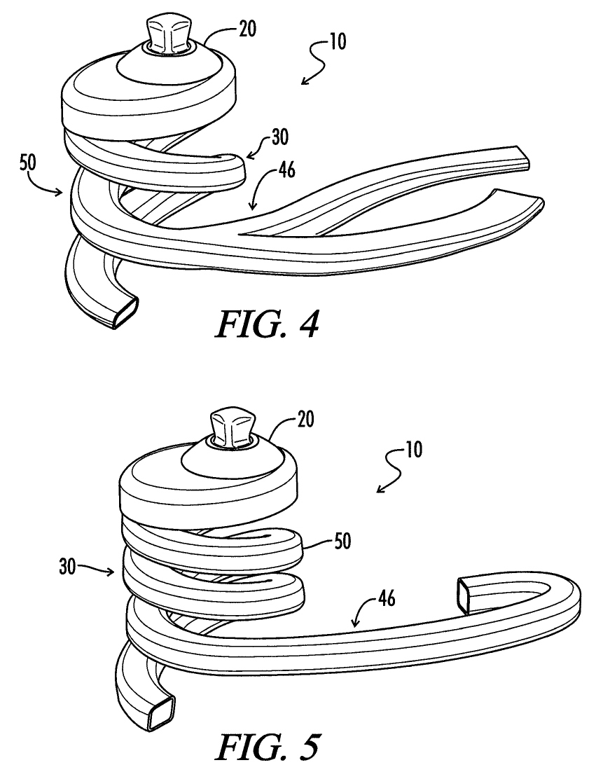

Turning then to alternative embodiments of the prosthetic foot according to the present invention, FIG. 4 depicts a prosthetic foot 10 with ankle plate 20 and both a heel spring 30 and forespring 50 descending helically substantially vertically beneath the ankle plate. Forespring 50 connects to an arch 46 to form a resilient keel portion, the forward part of which is divided into two hollow tubing sections. It will be seen that the cross sectional profile of the hollow tubing of FIG. 4 is nearly rectangular with slightly rounded corners. The hollow tubing of the prosthetic foot 10 of FIG. 5 has a nearly square cross section. Again, the heel spring 30 and forespring 50 are helically oriented in a nearly vertical direction beneath ankle plate 20 with heel spring 30 being connected to an arch section 46 to form a keel portion.

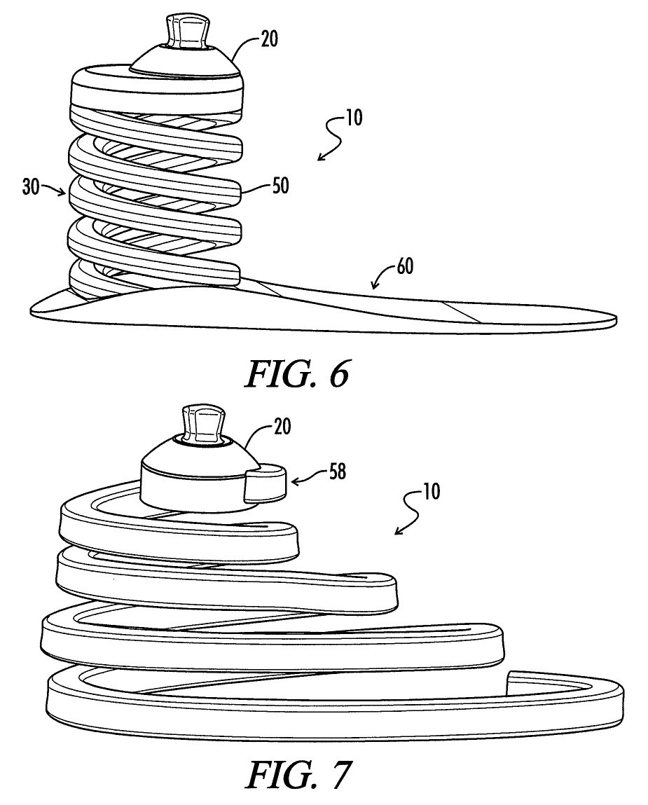

FIG. 6 shows another alternative embodiment of prosthetic foot 10 with the rear spring 30 and forespring 50 both connected to a flexible foot plate 60. The prosthetic foot 10 of FIG. 7 has an ankle plate 20 with a single spring 58 extending helically downward elliptically about a substantially vertical axis with each loop of the spring gradually increasing in length. This provides a low profile prosthetic foot of exceptional wire length.

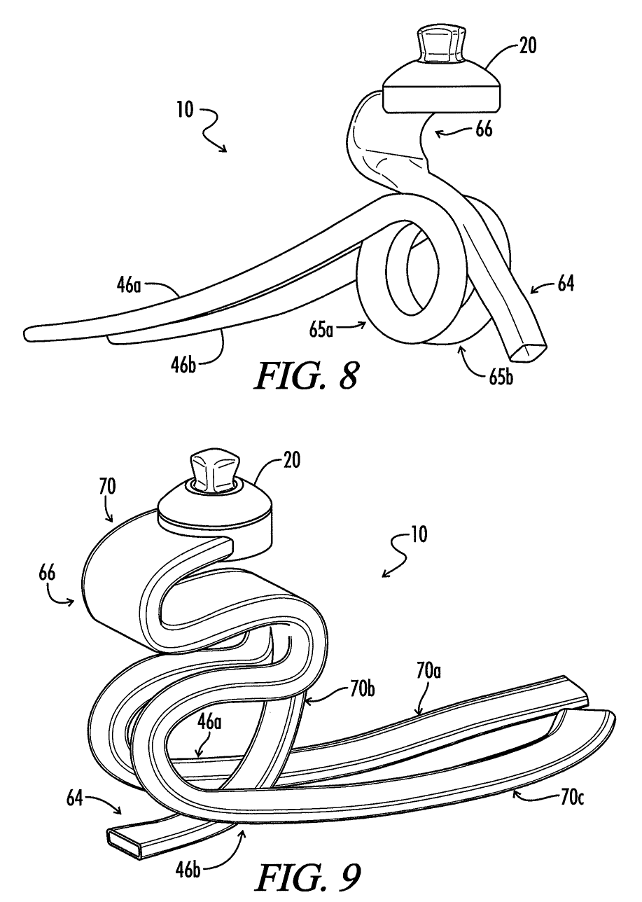

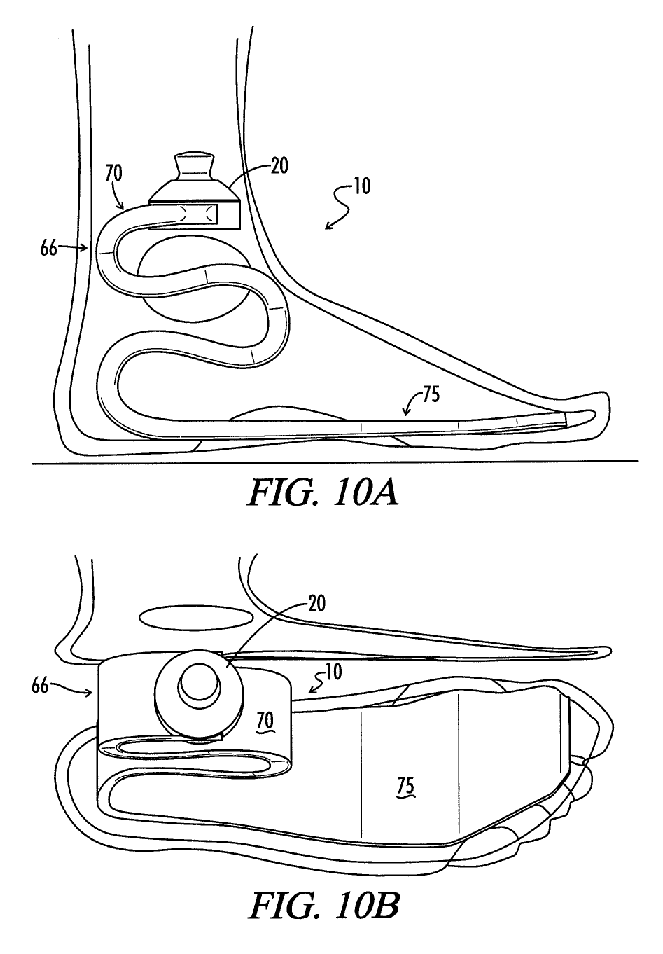

FIG. 8 depicts another prosthetic foot 10 with ankle plate 20 connected to a heel spring formed of bend 66 and rearward extending tube 64. The keel portion is formed of foresprings 65a, 65b and corresponding arch portions 46a and 46b. The helical springs 65a, 65b are curved about a horizontal axis through an arc of about 360 degrees in this embodiment. Curvature about a horizontal axis through arcs of at least about 120 degrees, 150 degrees, 180 degrees, 210 degrees, 240 degrees, 270 degrees, 300 degrees, 330 degrees and more may also produce desirable results. In the embodiment of FIG. 9, a hollow composite tubular section that may advantageously be fabricated in a multi-cellular fashion comprising tubes 70a, 70b, and 70c extends from ankle plate 20 through first bend 66 to the second bend where the heel spring element 64 separates while the arch components 46a, 46b extend through a third bend and forward to complete a keel portion of the foot. In FIG. 10A, another relatively broad hollow tube 70, which again may be of multi-cellular structure, extends from ankle plate through first bend 66, second bend and third bend before extending forward in a foot plate portion 75.

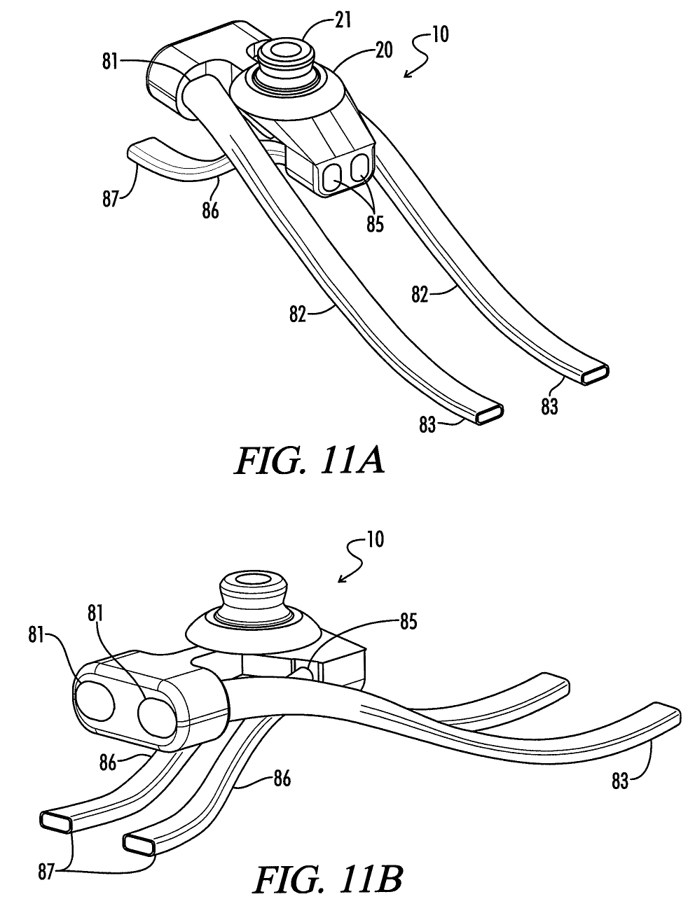

FIG. 11A discloses an alternative prosthetic foot 10 with ankle plate 20 having attachment point 21, two forwardly oriented tubing attachment points 81 to secure forwardly extending composite tubes 82 having wave shapes extending to flat forward resting points 83. Also, two rearwardly extending attachment points 85 secure rearwardly extending composite tubes 86 having wave shapes extending to flat rear resting points 87. The use of pairs of forwardly extending and rearwardly extending tubes facilitates a balanced design, however, alternate constructions with a single tube or more than two tubes oriented in a direction are possible.

All publications, patents, and patent documents are incorporated by reference herein as though individually incorporated by reference. Numerous alterations of the structure herein disclosed will suggest themselves to those skilled in the art. However, it is to be understood that the present disclosure relates to the preferred embodiment of the invention which is for purposes of illustration only and not to be construed as a limitation of the invention. All such modifications which do not depart from the spirit of the invention are intended to be included within the scope of the appended claims.

We claim:

1. A prosthetic foot comprising:
(a) a mounting element securable to a lower limb prosthesis, which is in turn securable to the residual limb;
(b) a first hollow composite biasing structure at a heel portion of the foot attached to the mounting element of the foot.

2. The prosthetic foot of claim 1 wherein the hollow composite biasing structure is curved about a vertical axis.

3. The prosthetic foot of claim 1 wherein the hollow composite biasing structure is curved about a horizontal axis through an arc of at least 120 degrees.

4. The prosthetic foot of claim 1 further comprising a second hollow composite biasing structure connected to the mounting element and forming a keel extending forward of the first hollow composite housing structure.

5. The prosthetic foot of claim 1 wherein the hollow composite biasing structure is a helical spring.

6. The prosthetic foot of claim 1 wherein the hollow composite structure is a composite wall defining a lumen extending in an axial direction.

7. The prosthetic foot of claim 6 wherein the composite wall comprises at least a fabric layer oriented at a 45 degree angle to the axial direction on all of the top, bottom and sides of a portion of the wall.

8. The prosthetic foot of claim 1 wherein at least some portion of the loading of the hollow composite biasing structure is in torsion.

9. The prosthetic foot of claim 4 wherein at least some portion of the loading of the keel is in torsion.

10. The prosthetic foot of claim 1 wherein the hollow composite structure is made from fibers selected from the group of carbon, aramid, glass, boron, polyvinyl alcohol, ceramic, piezoelectric and fiberglass.

11. A prosthetic foot comprising:
(a) an ankle plate securable to a residual limb and having a first mounting point for a heel spring and a second mounting point for a forespring;
(b) a heel spring formed of hollow composite tubing extending from the first mounting point; and
(c) a forespring formed of hollow composite tubing extending from the second mounting point.

12. The prosthetic foot of claim 11 wherein at least one of the heel spring and the forespring is curved about a vertical axis.

13. The prosthetic foot of claim 11 wherein at least one of the heel spring and the forespring is curved about a horizontal axis through an arc of at least 120 degrees.

14. The prosthetic foot of claim 11 wherein a forward extending keel comprises the forespring.

15. The prosthetic foot of claim 11 wherein at least one of the heel spring and the forespring is a helical compression spring.

16. The prosthetic foot of claim 11 wherein the hollow composite tubing has a cross-section selected from the group of round, oval, elliptical and rectangular.

17. The prosthetic foot of claim 11 wherein a wall of the hollow composite tubing comprises at least a fabric layer oriented at a 45 degree angle to the axial direction of the tubing all of the top, bottom and sides of a portion of the wall.

18. The prosthetic foot of claim 11 wherein at least some portion of the loading of the heel spring is in torsion.

19. The prosthetic foot of claim 11 wherein at least some portions of the loading of the forespring spring is in torsion.

20. The prosthetic foot of claim 14 further comprising a lateral longitudinal support element extending between the heel spring and the keel.
